(19) United States
(12) Patent Application Publication
(10) Pub. No.: US 2009/0220542 A1
(43) Pub. Date: Sep. 3, 2009

(54) VACCINE COMPRISED SPECIFICALLY OF PROTEIN SUBUNITS OF HUMAN IMMUNODEFICIENCY VIRUS'S GLYCOPROTEIN 120 PROBE TO PREVENT AND TREAT AN INFECTION CAUSED BY THE HUMAN IMMUNODEFICIENCY VIRUS

(76) Inventors: LANE BERNARD SCHEIBER, Annandale, VA (US); LANE BERNARD SCHEIBER, II, Grosse Ile, MI (US)

Correspondence Address: LANE B. SCHEIBER, 7864 OAK RIVER DRIVE, GROSS ILE, MI, 48138, US

(21) Appl. No.: 12/040,898

(22) Filed: Mar. 2, 2008

(51) Int. Cl. A61K 39/21; A61P 31/18

(52) U.S. Cl. 424/208.1

(57) ABSTRACT

The human immunodeficiency virus poses a significant threat to the health and well being of the world's population. Current strategies utilized to eradicate this deadly pathogen have not been effective. A vaccine comprised solely of protein subunits of the glycoprotein 120 probe as the active ingredient, can be effective in stimulating an individual's immune system to repel an HIV infection. The protein subunit of the glycoprotein 120 probe extends from the surface of HIV and is the unique identifier of an HIV virion. When protein subunits of HIV's glycoprotein probe are exclusively presented to the immune system, the antibodies generated will neutralize the glycoprotein 120 probes located on the surface of HIV virions, such that the virus's virions then are incapable of engaging a T-Helper cell and thus the infectious threat posed by HIV is averted.

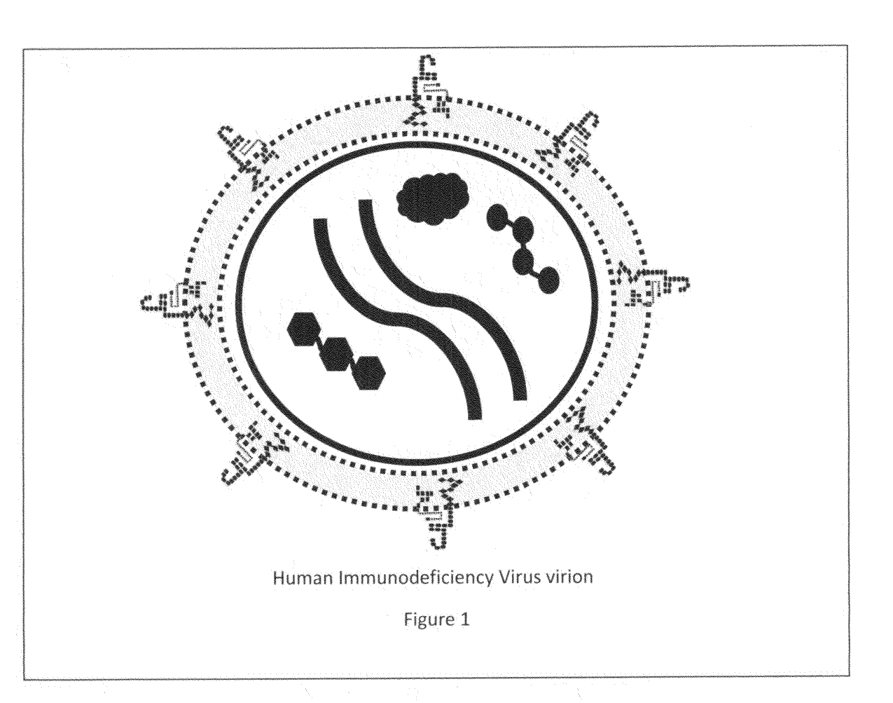

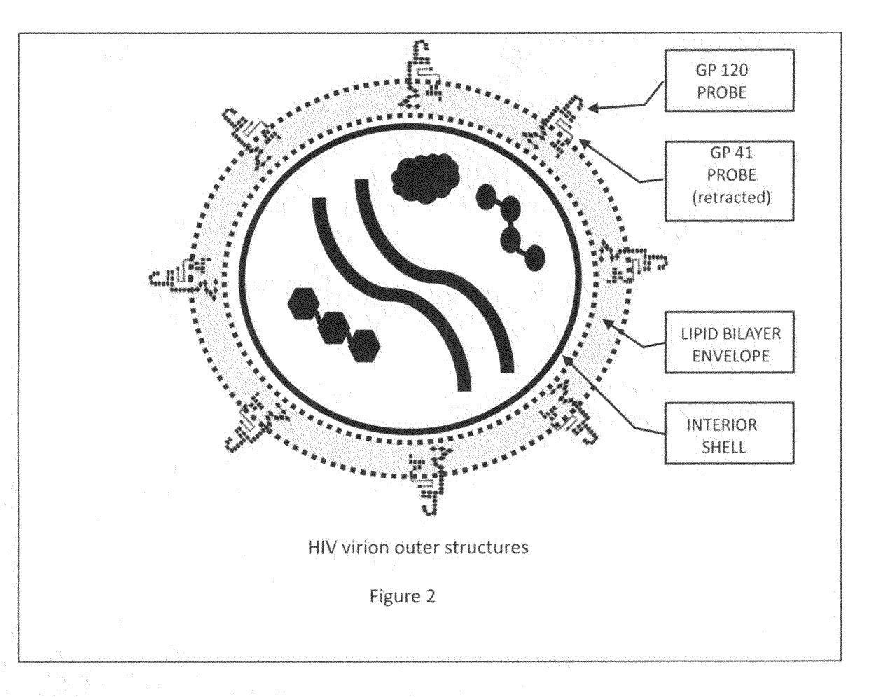

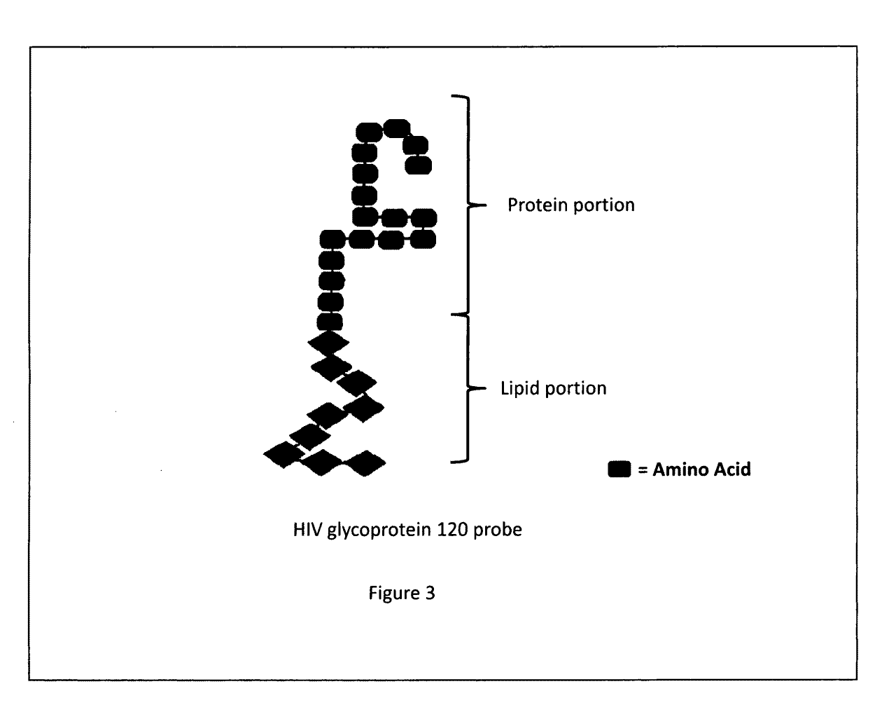

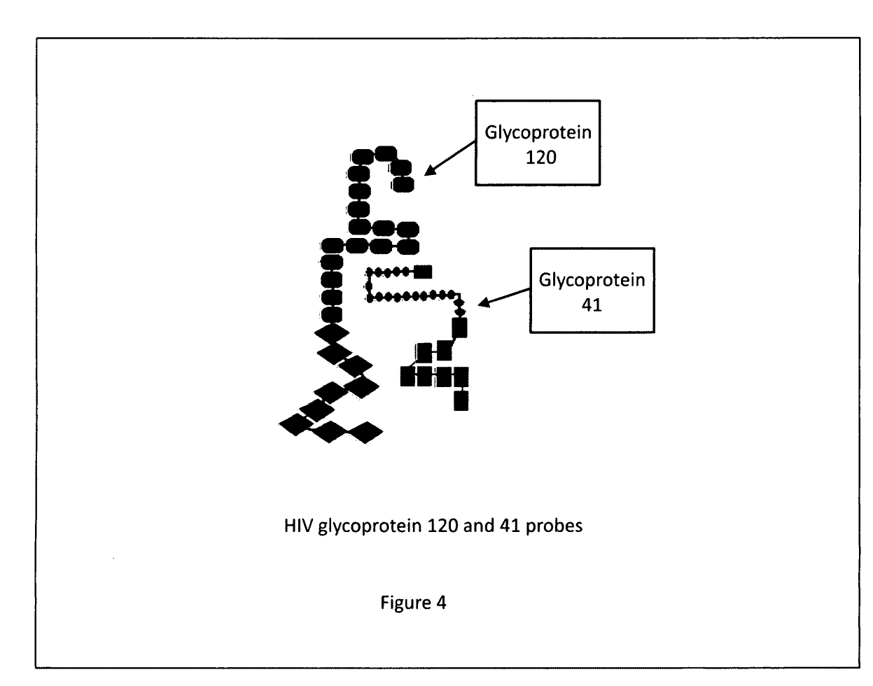

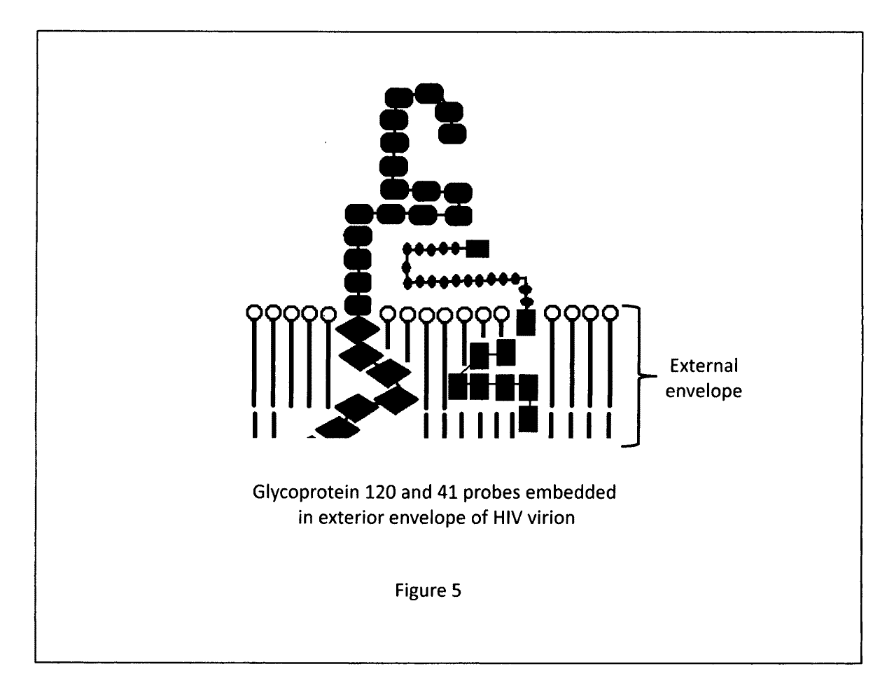

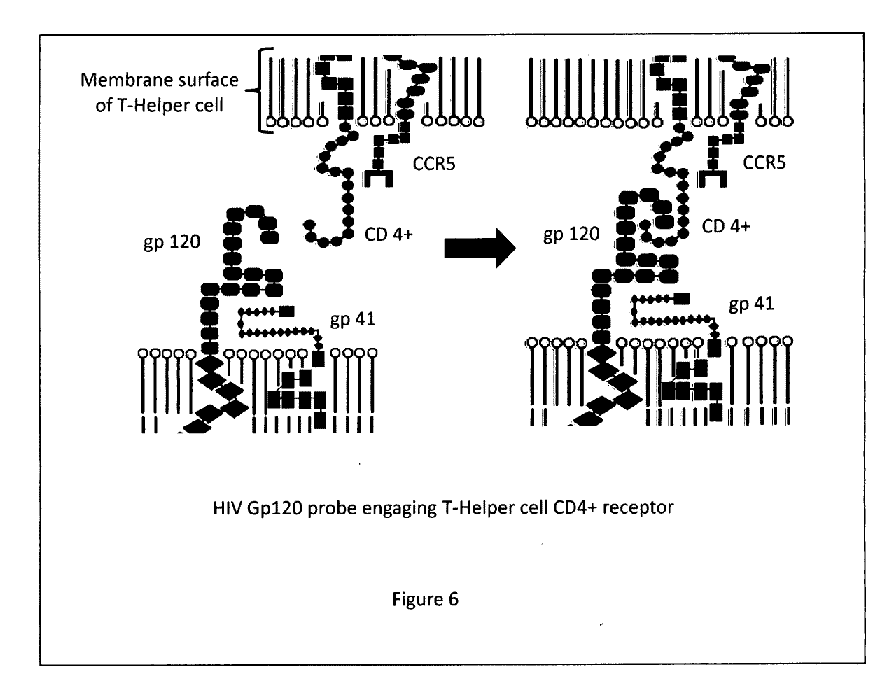

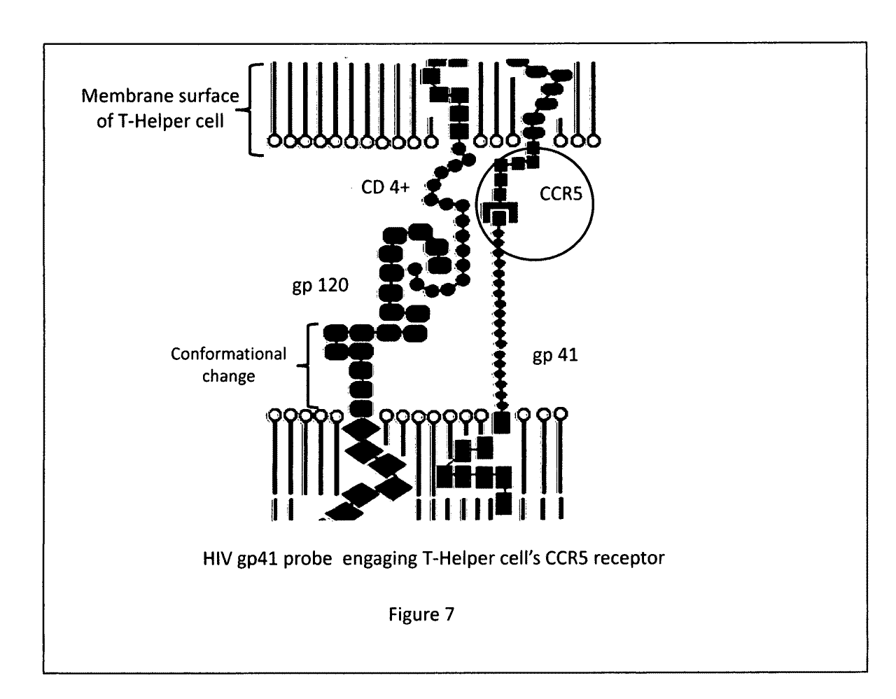

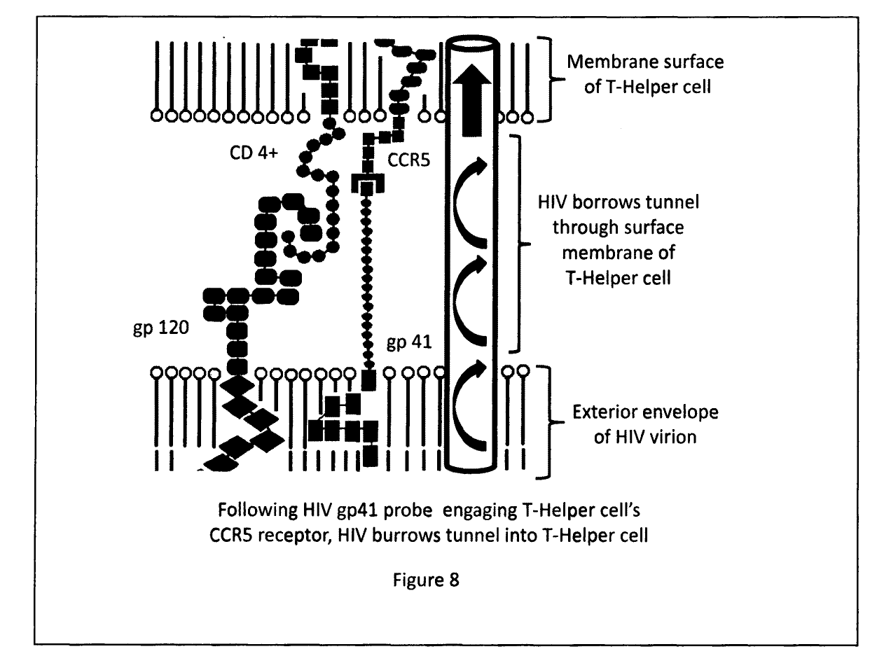

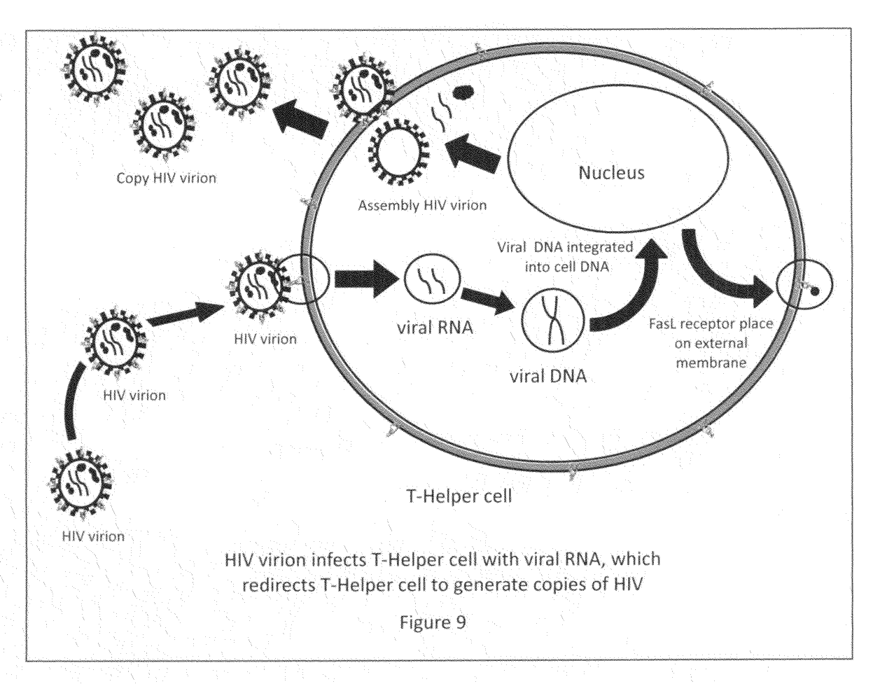

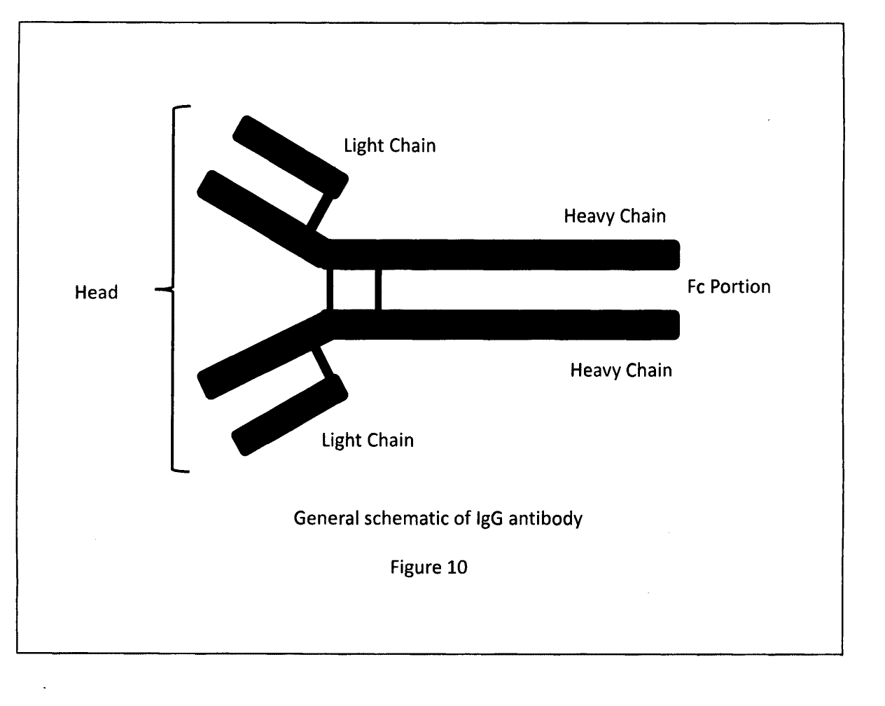

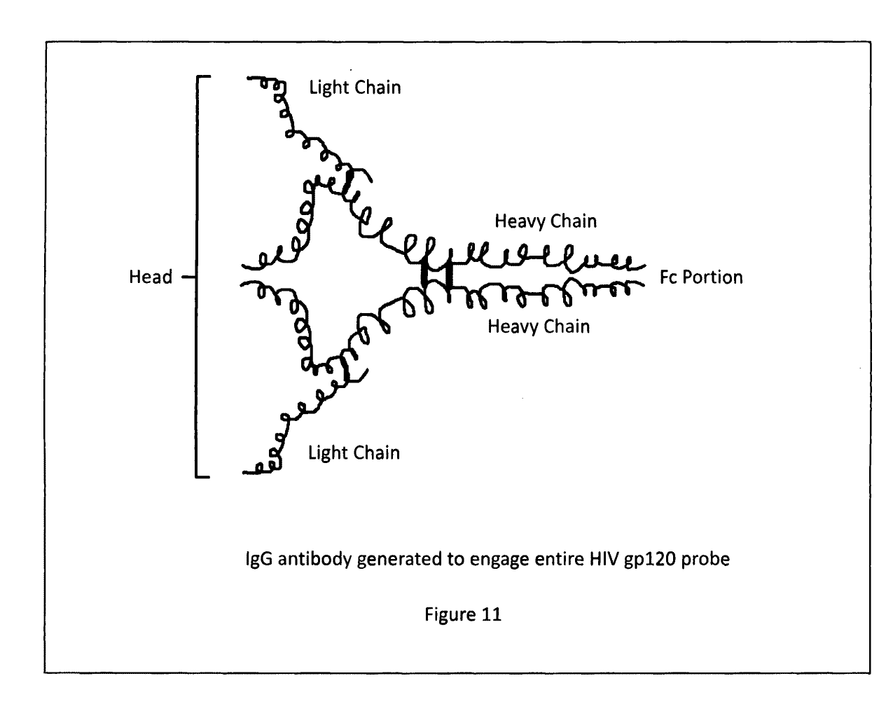

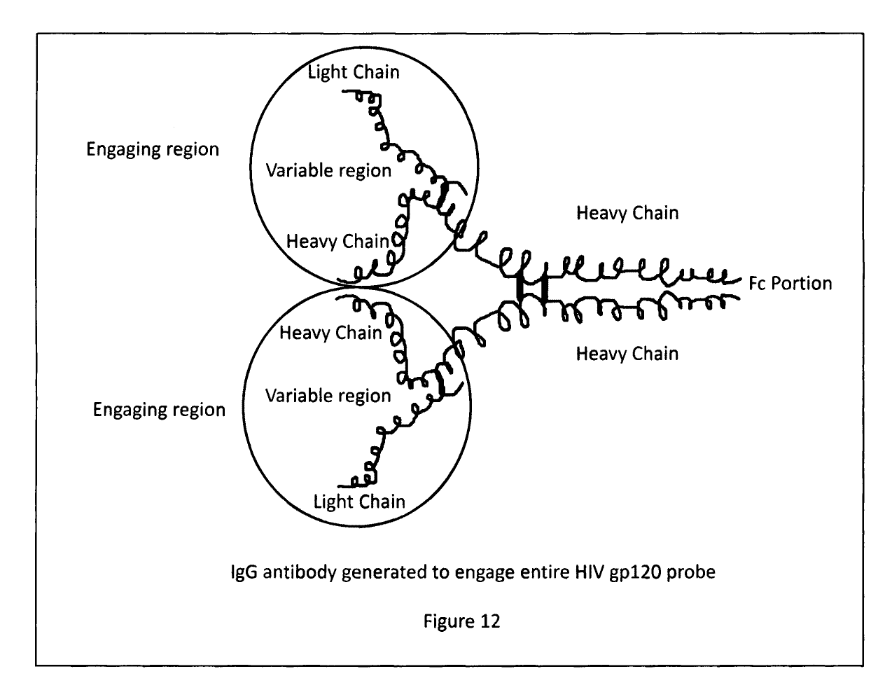

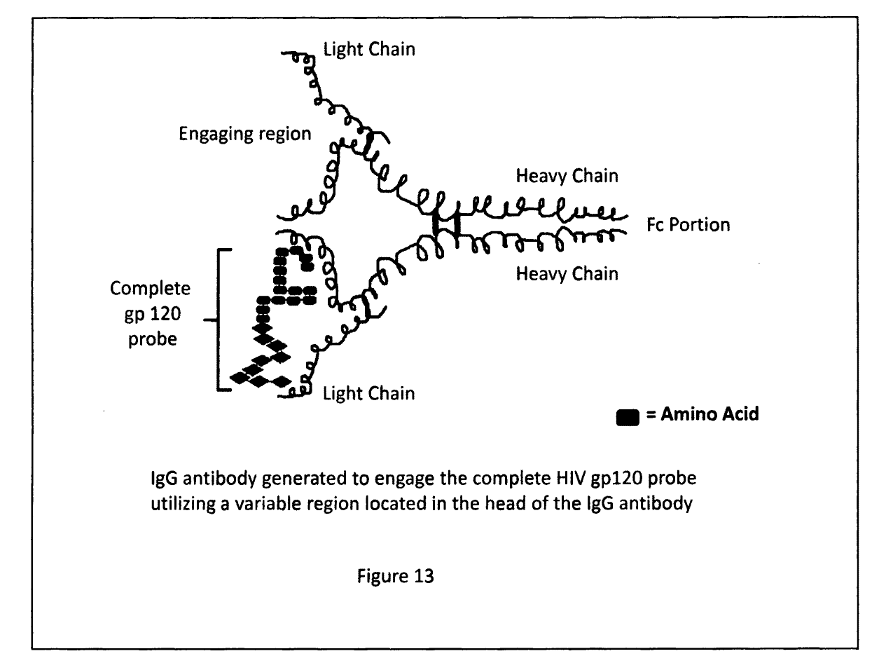

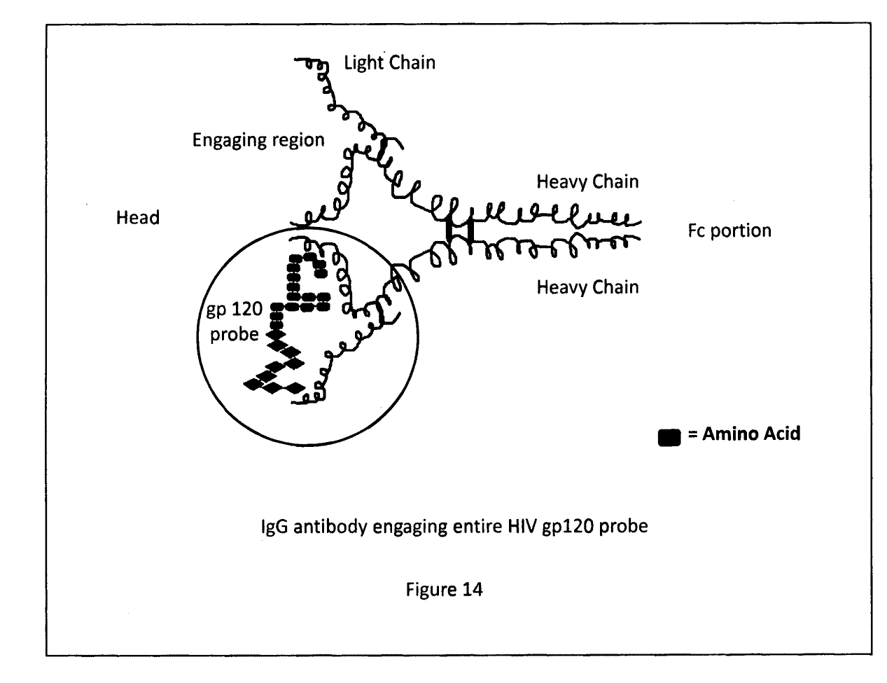

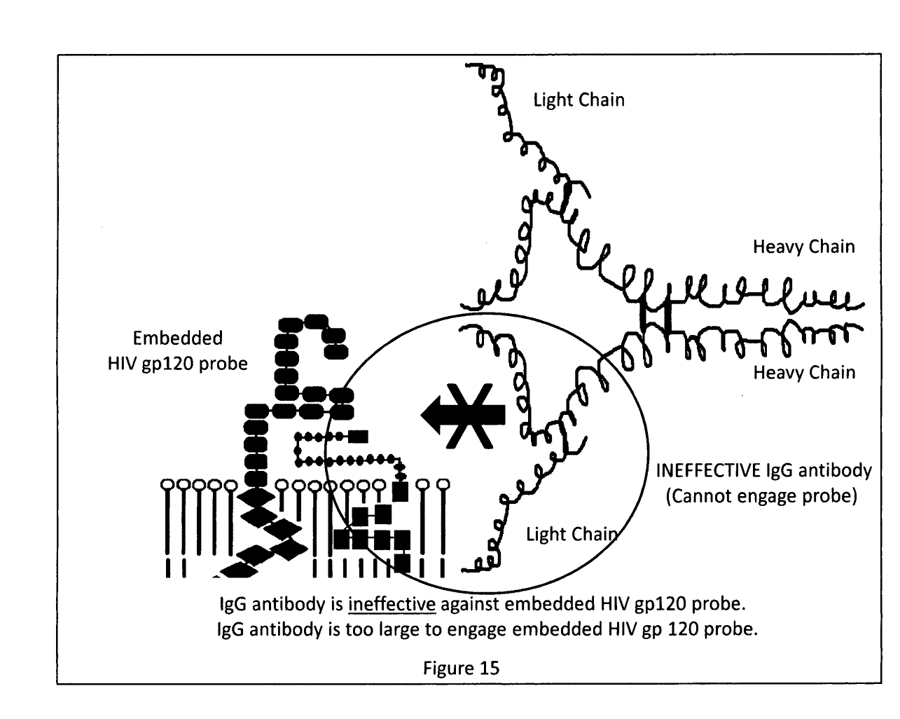

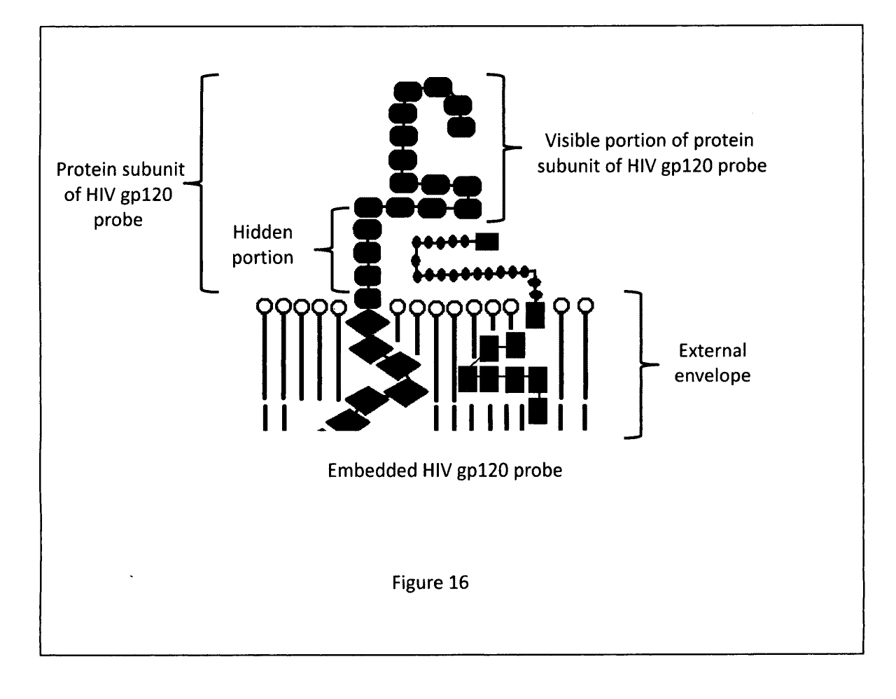

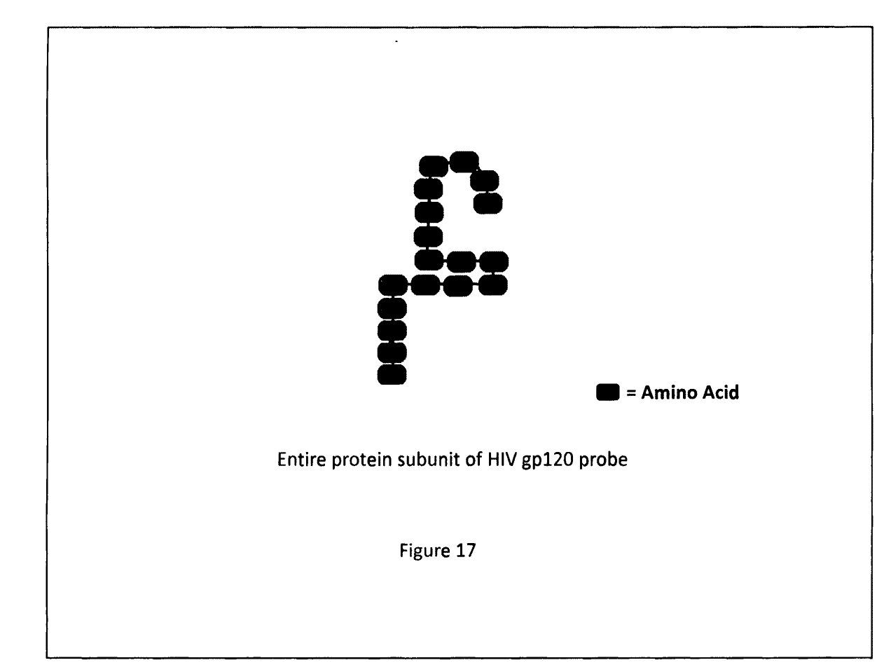

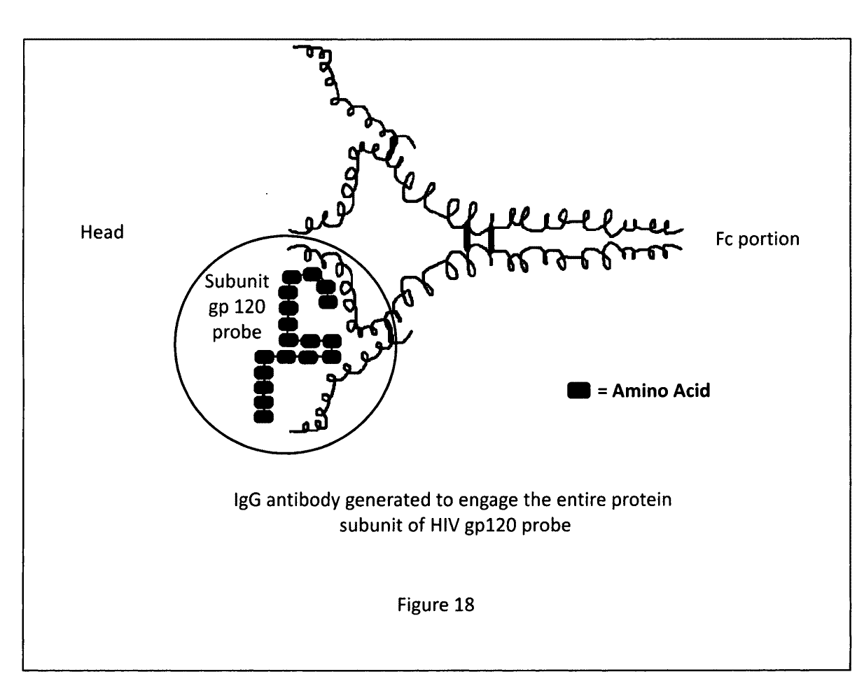

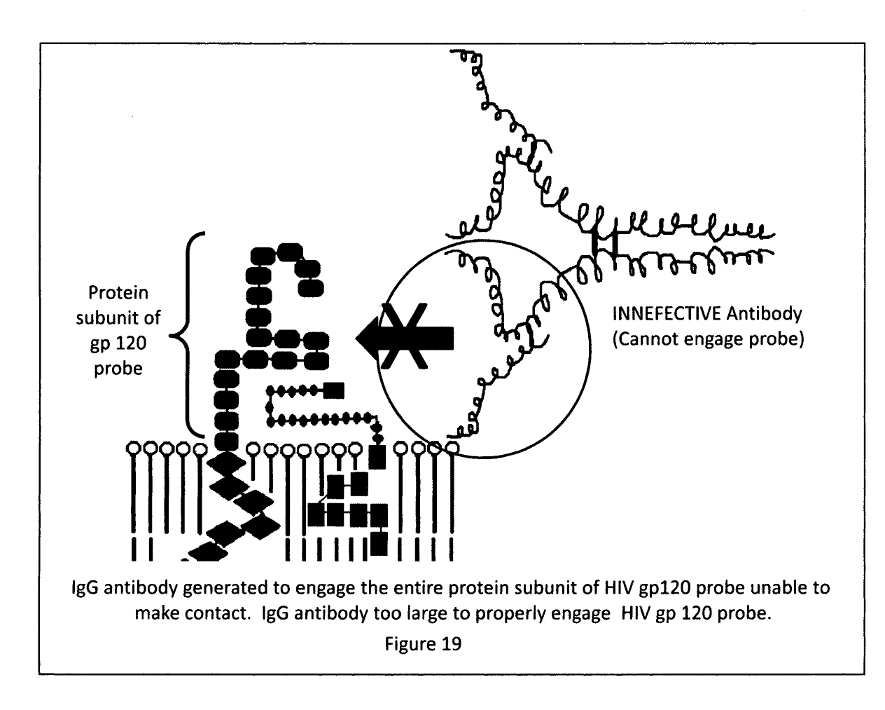

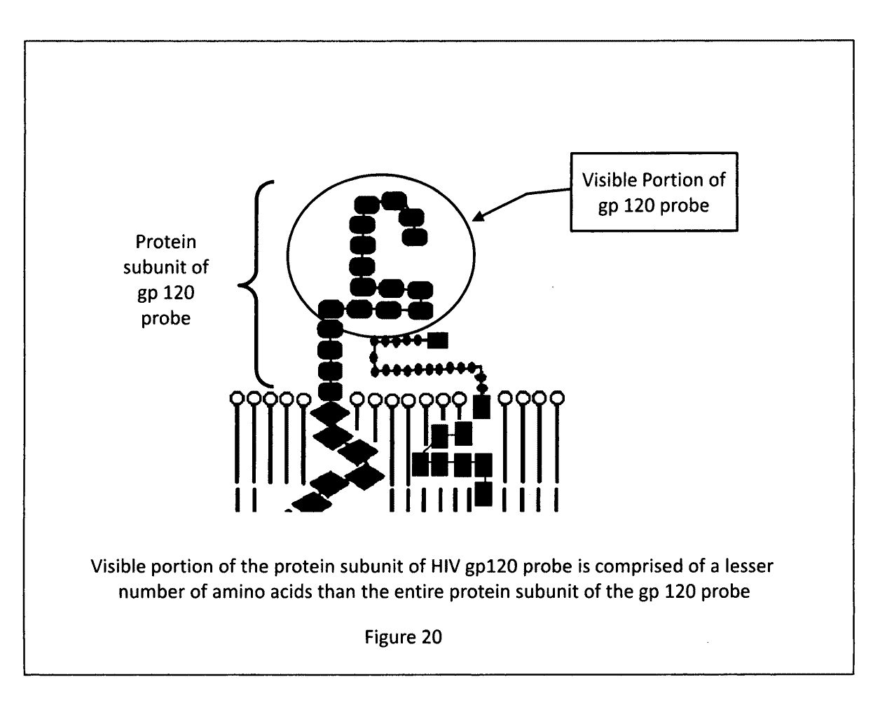

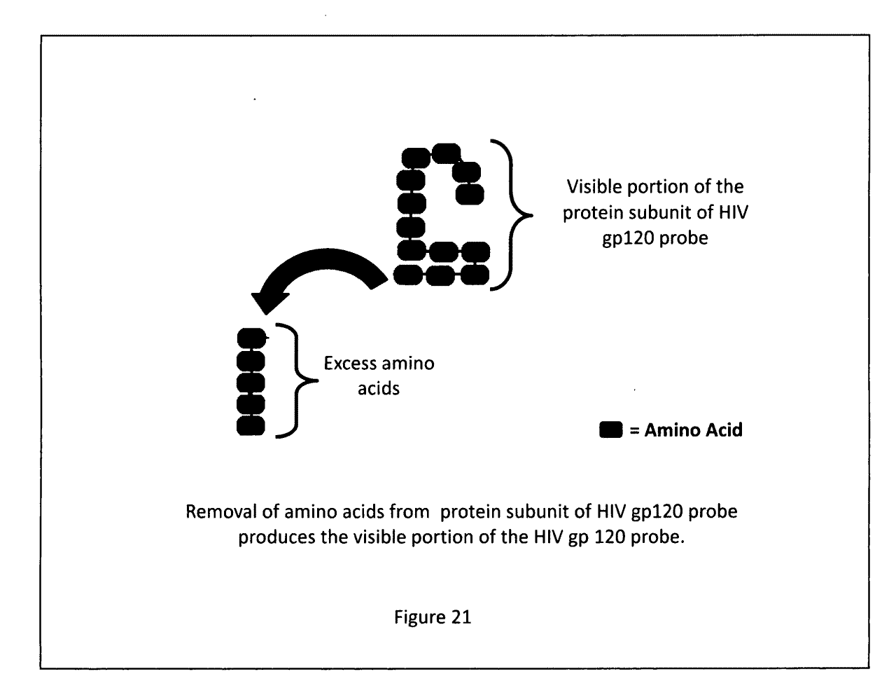

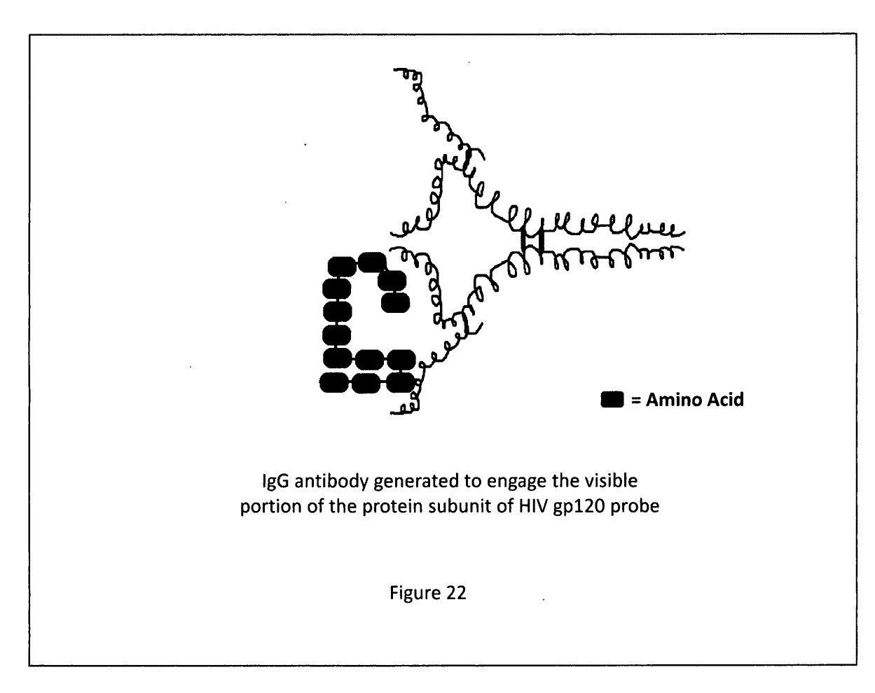

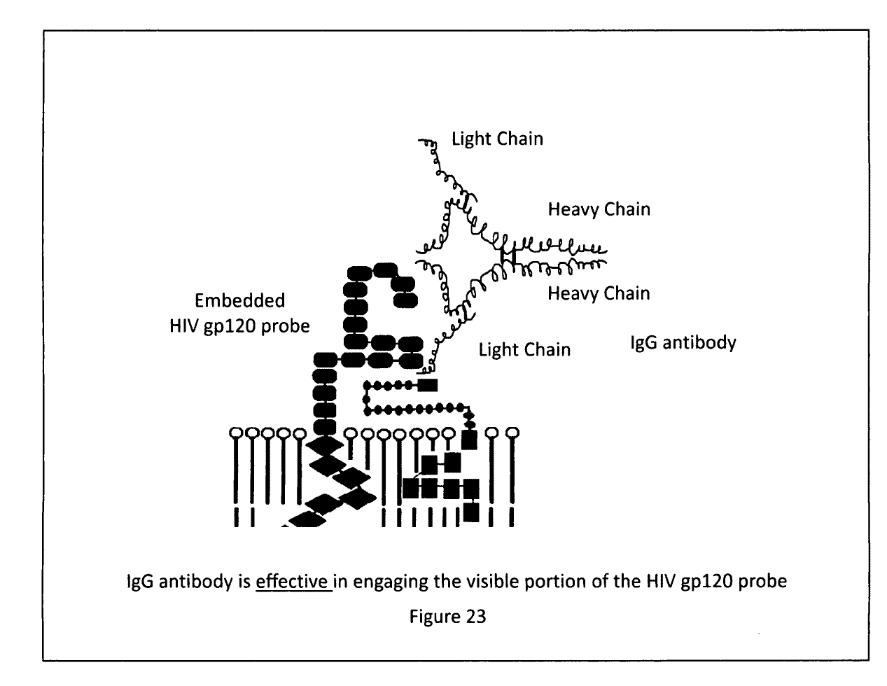

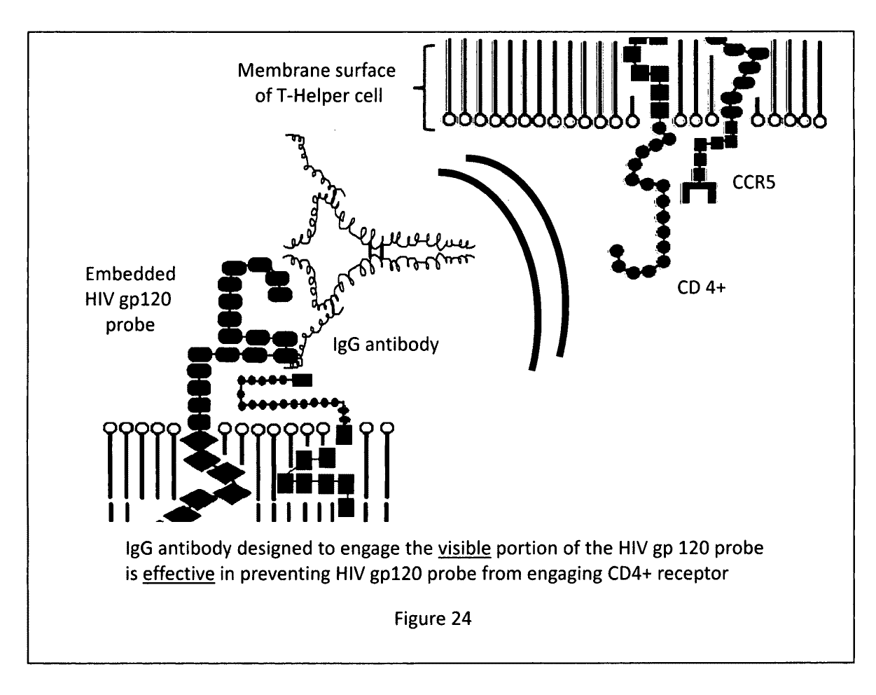

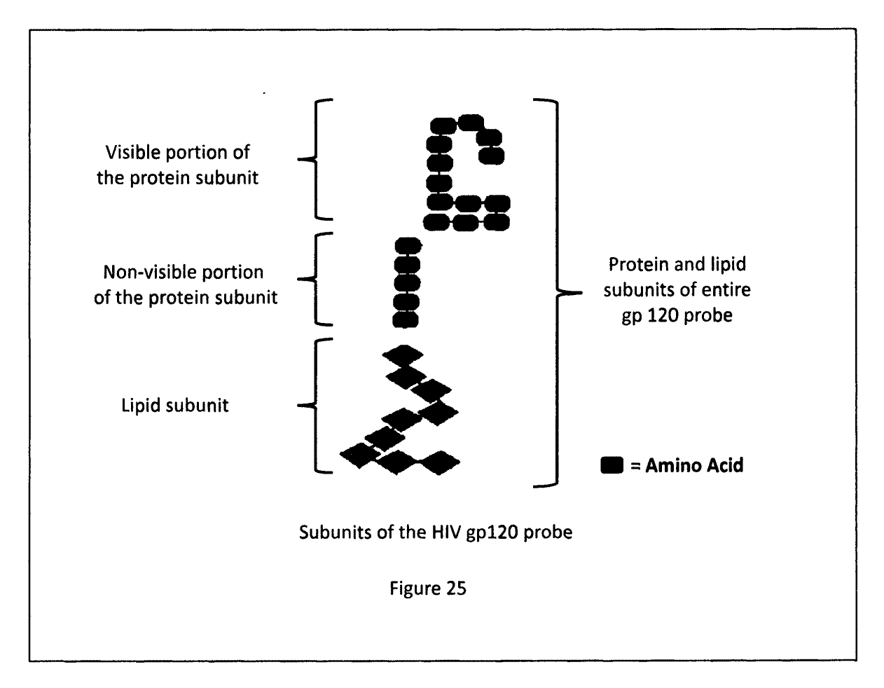

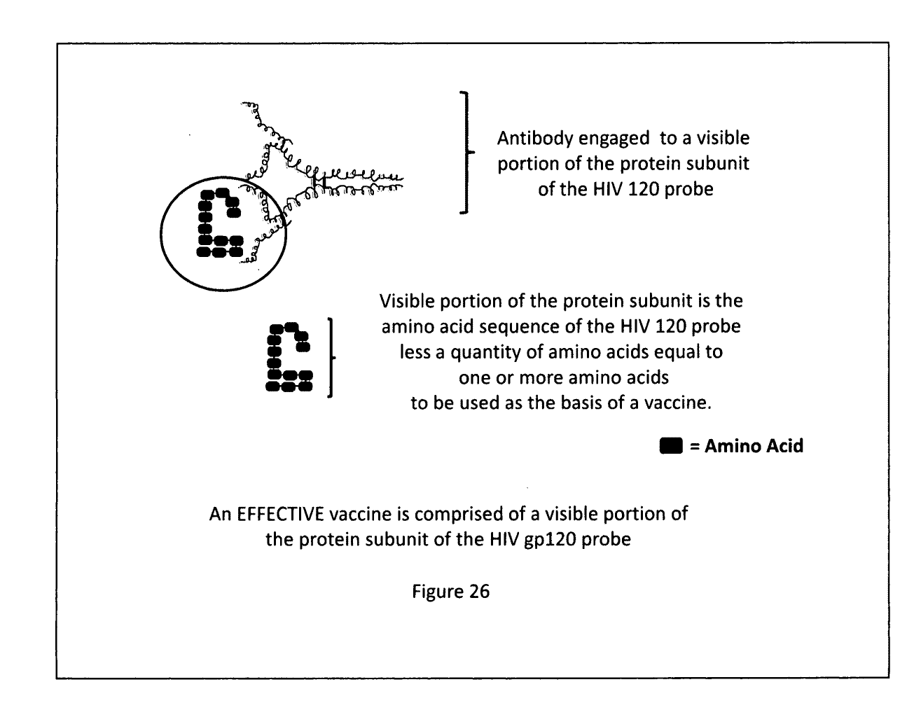

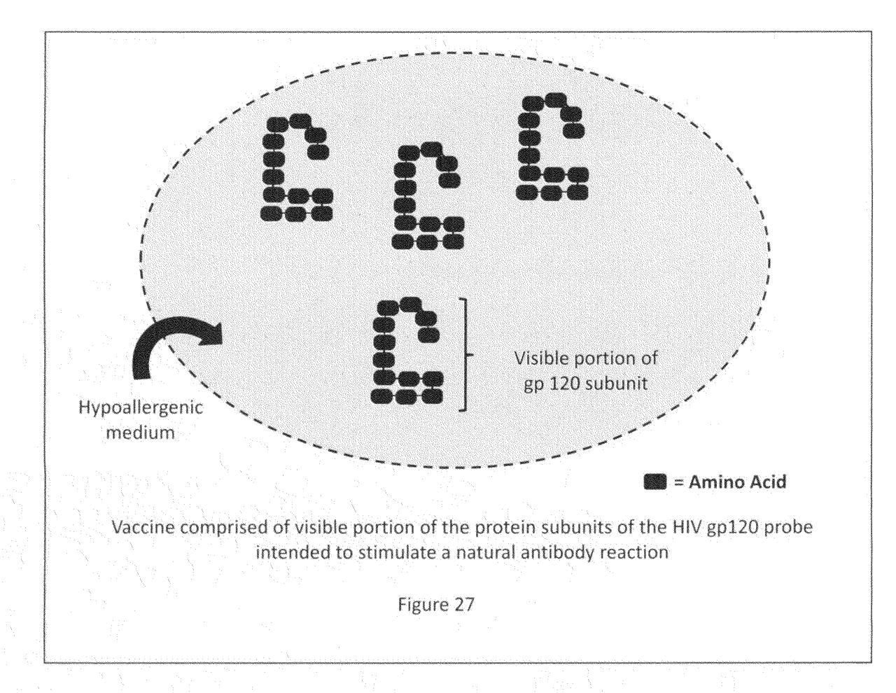

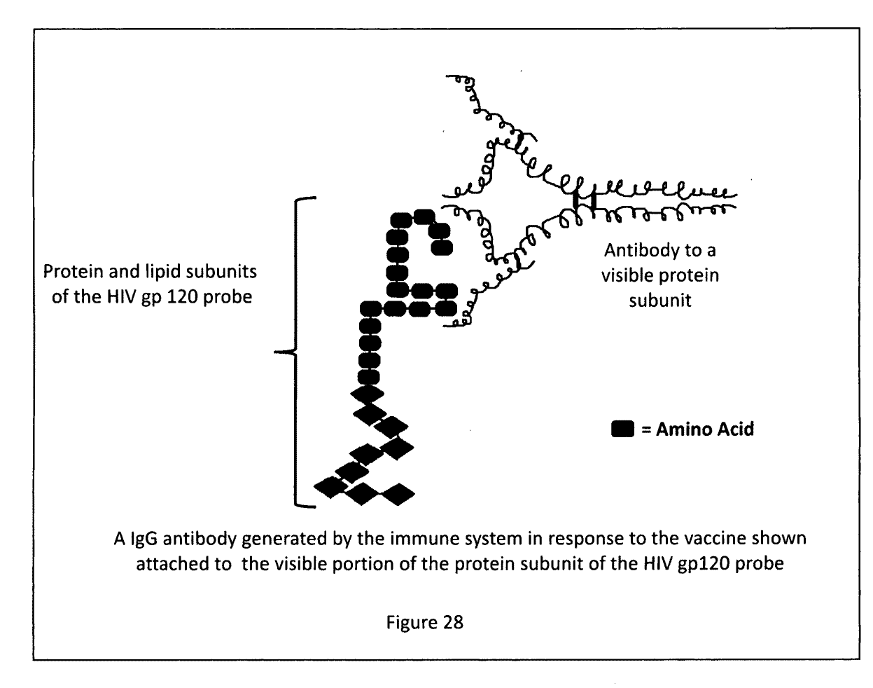

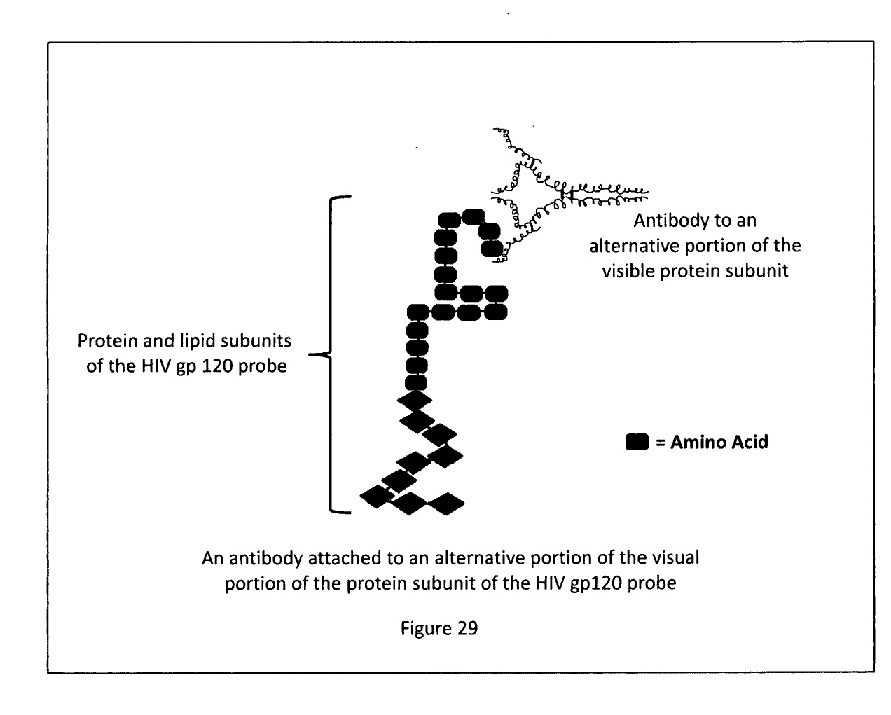

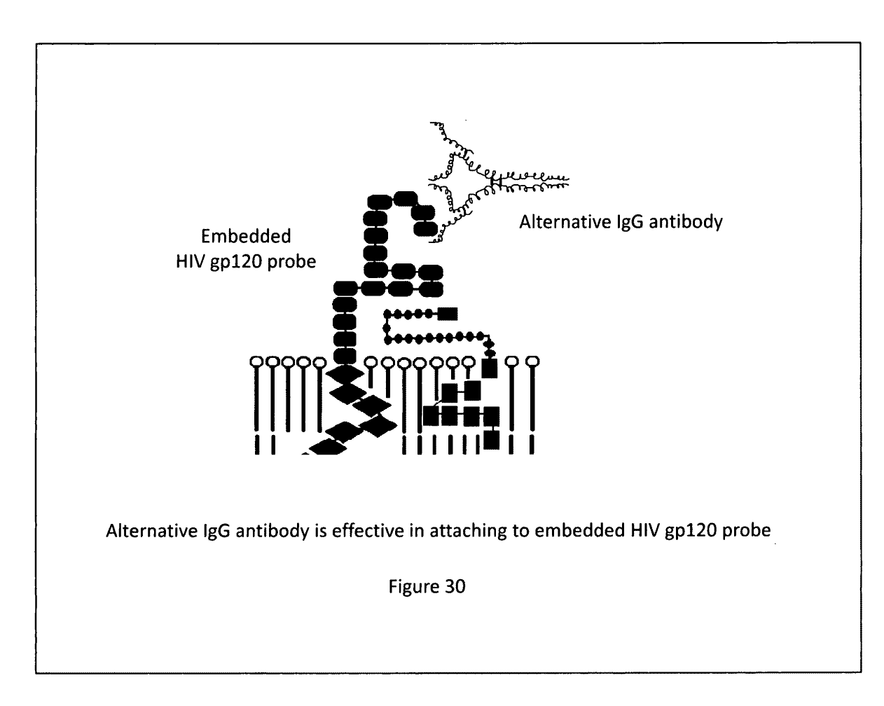

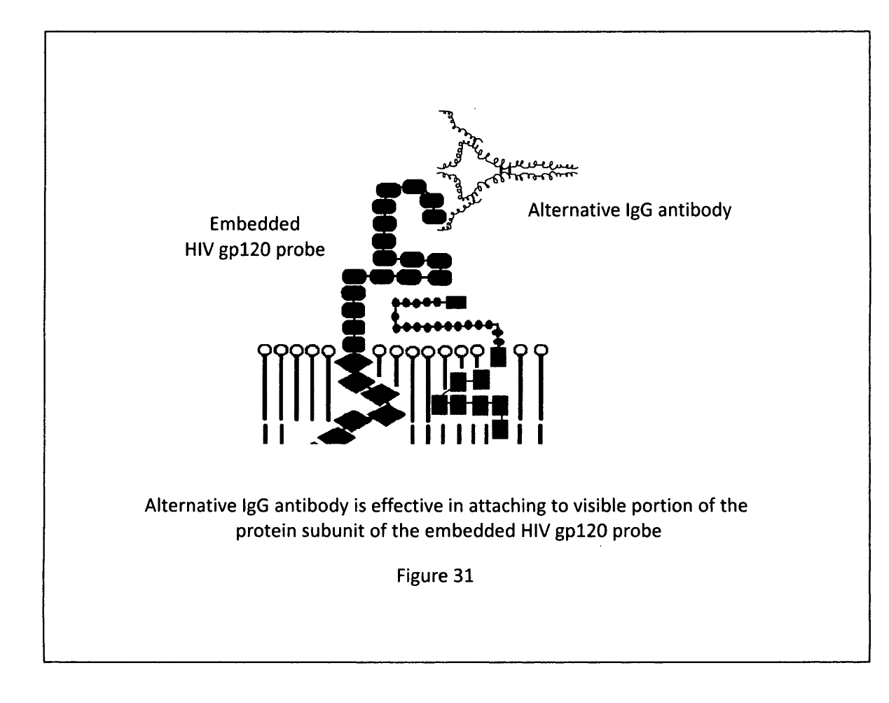

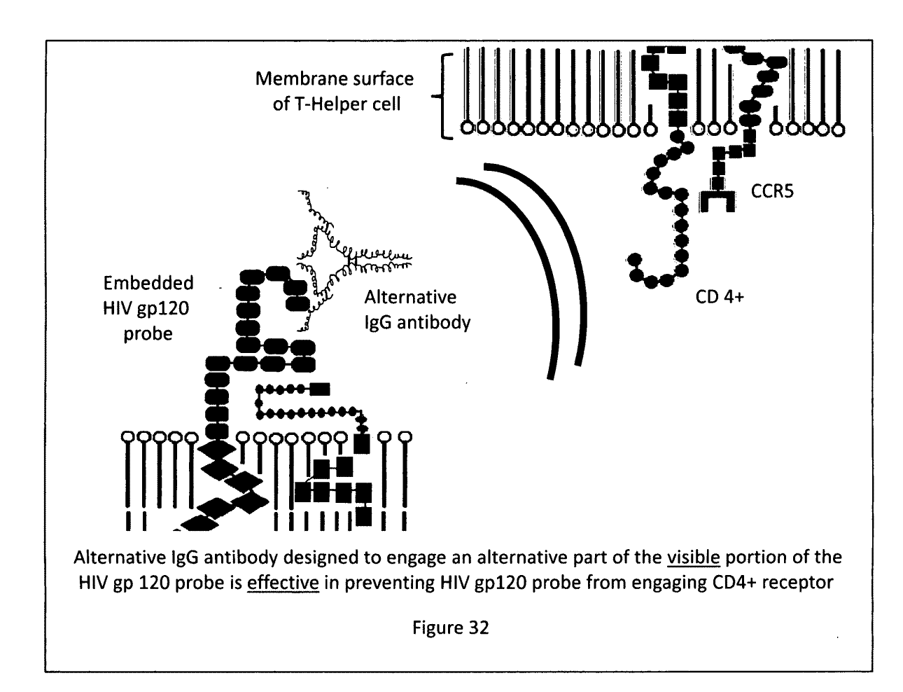

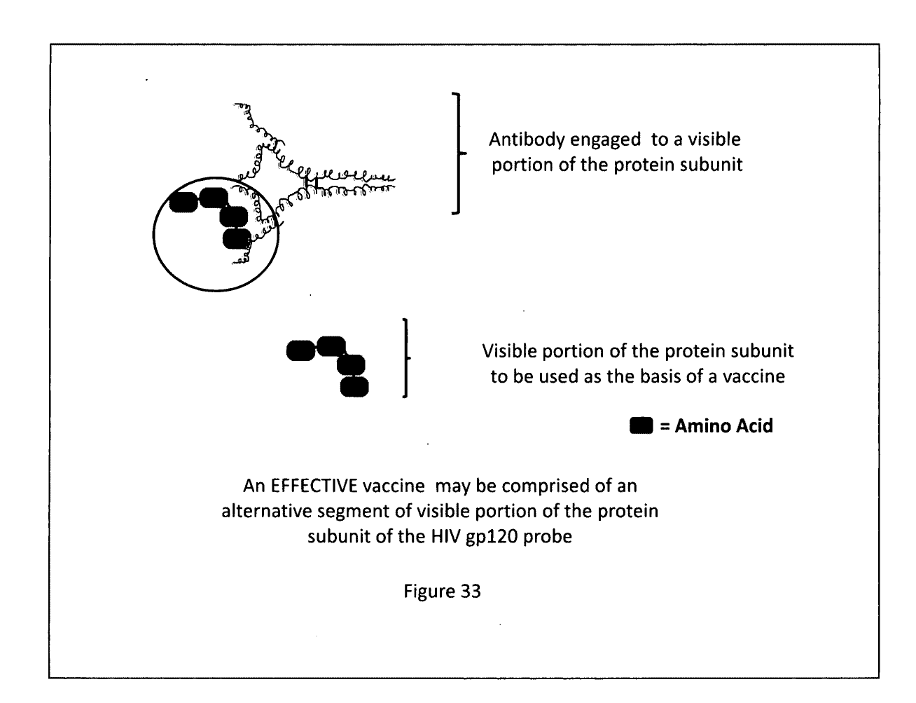

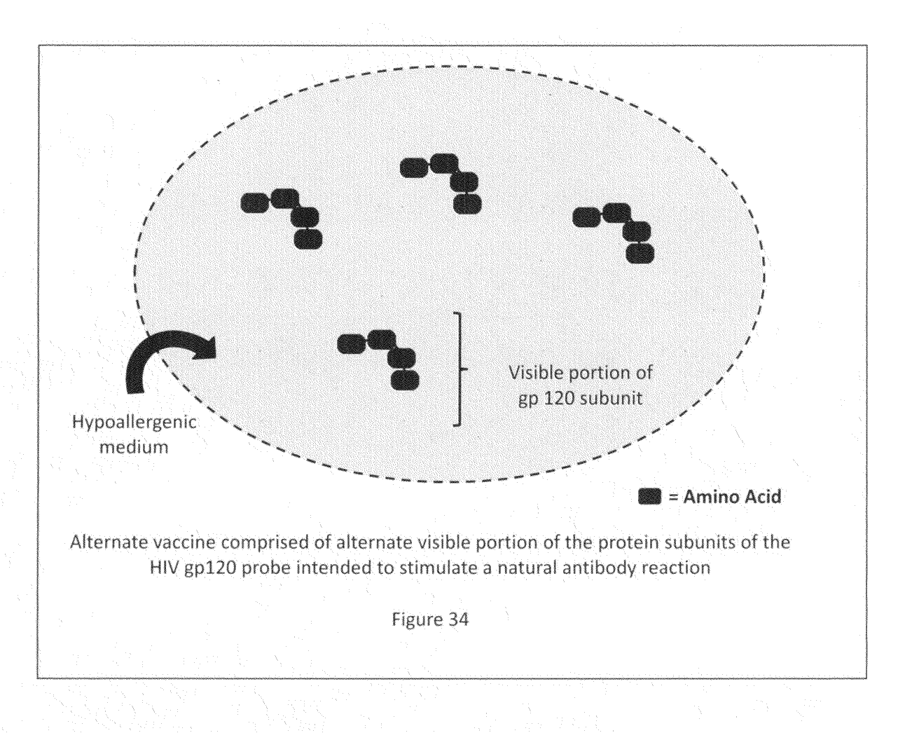

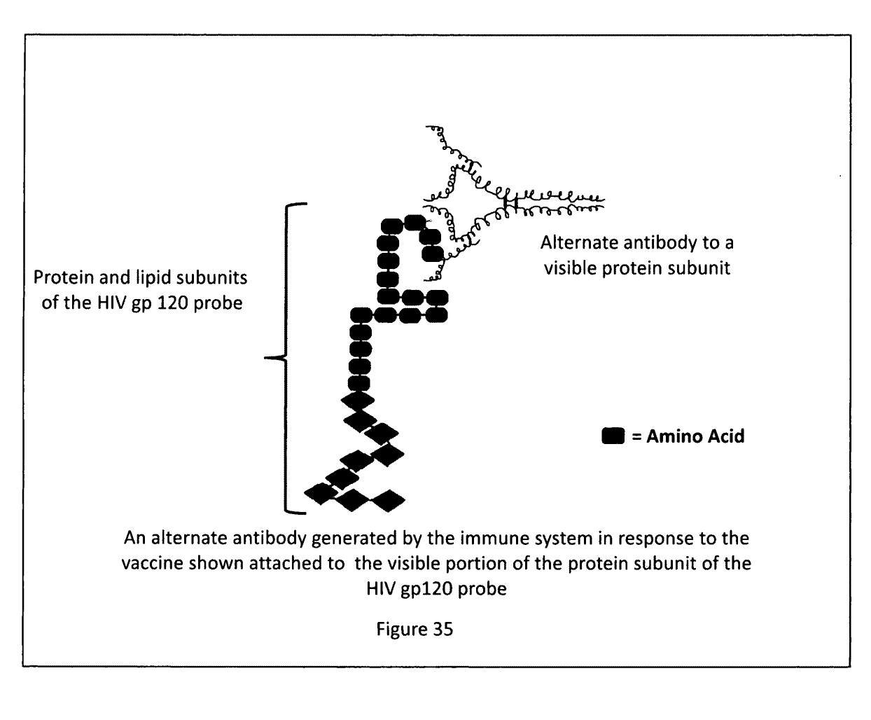

### CROSS-REFERENCED TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

®2008 Lane B. Scheiber and Lane B. Scheiber II. A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device introduced into the body, which is intended to cause an immune response against the human immunodeficiency virus to prevent or treat an infection caused by the human immunodeficiency virus.

2. Description of Background Art

According to the Center for Disease control, in the United States it is estimated that as of December 2004 their have been 944,306 people infected with the Human Immunodeficiency Virus (HIV), and 56% of these individuals have died. It is estimated there are at least 40,000 new cases of individuals infected with HIV in the United States per year with 25% of patients unaware they have contracted the virus. Further, it has been estimated by the World Heath Organization that HIV infects 33 million people worldwide. After over twenty years of research and investigation, eradicating the ever-growing global humanitarian crisis posed by the HIV remains an elusive goal for the medical community.

The Human Immunodeficiency Virus is ingeniously configured as well as a deadly virus. Viruses, in general, have been difficult to contain and eradicate due to their being obligate parasites and the fact they tend not to carry out biologic functions outside the cell the virus has targeted as its host. An intact, individual form of a virus, as it exists outside the boundaries of a host cell, is generally referred to as a ‘virion’. The human body's immune system possesses innate mechanisms to repel viral infections once such a pathogen breaches the perimeter defenses and is recognized as an invader. If a cell is determined to be infected with a virus, neighboring cells may generate a defense response that causes neighboring cells to resist infection or if neighboring cells become infected, such cells shuts down biologic processes the virus might attempt to utilize for the purpose of replication. HIV virions possess several attributes that make them especially elusive, circumventing the immune system's routine defensive measures.

Bacteria generally have posed a much easier target for the medical community to eradicate compared to viral infections. Bacteria generally live and reproduce outside animal cells. When a white cell encounters a bacterium, the white cell engulfs the bacterium, encapsulates the pathogen, processes the identification of the pathogen and kills the pathogen utilizing acids and destructive enzymes. The white cell then alerts the B-cells of the immune system as to the identity of the intruding pathogen. A subpopulation of B-cells is created. This subpopulation of B-cells is dedicated to producing antibodies directed against the pathogen the circulating white cell encountered and identified. A variety of other cells, such as, dendritic cells, macrophages and circulating B-cells may also engage a pathogen and stimulate an immune response. Antibodies, generated by B-cells, traverse the blood and body tissues in search of the bacteria they were designed to repel. Antibodies attach to bacteria, coating the surface of bacteria, attempting to punch holes through a bacterium's cell wall and signal the cells comprising the immune system to the presence of the bacterium.

Viruses pose a much different challenge to the body's defense system than do bacteria. Since viruses generally do not carry out biologic processes outside their host cell, a virus virion can be destroyed, but there are no on-going internal biologic life-sustaining processes to terminate. A virus is simply comprised of one or more external shells, one or more segments of genetic material carried inside the core of the innermost shell, and many viruses contain one or more enzymes to assist in the replication of the virus. Antibodies can coat the exterior of a virus to make it easier for the white cells in the body to identify the pathogen, but the action of punching holes in the virus's external shell does not terminate any life functions. Many viruses only briefly circulate in the blood and tissues of the body, thus exist for only a limited time as an exposed and vulnerable entity.

Viruses utilize exterior probes to hunt down a cell in the body that will act as an appropriate host so that the virus can engage in the task of replicating of itself. Once the virus has found a proper host cell, the virus inserts its genome and any enzymes it carries into the host cell. The virus's genetic material takes command of cellular functions and the virus's genetic material diverts the host cell biologic machinery from normal cellular functions to engaging in constructing copies of the virus.

Once the virus has infected a host cell, the virus is in effect shielded from the body's immune system defense mechanisms by the cell membrane of the host cell. A virus which has infected a host cell, is generally only represented as ‘genetic information’ that often becomes intimately incorporated into the host cell's own DNA. Often, following the action of a virus's virion infecting a cell in the body, the presence of the virus can only be eradicated if the host cell is destroyed. Antibodies are generally designed to engage a bacteria or a virus and not to attack the naturally occurring tissues found in the body. Circulating white cells and the immune cells comprising lymph nodes and the immune cells comprising the spleen may or may not recognize that a cell, which has become a host for a virus, is carrying a virus's genetic material and is infected with a virus. If the immune system fails to identify a cell that has become infected with a virus, the virus's genetic material can proceed to force the infected cell to make copies of the virus. Since a virus is in essence simply a segment of genetic material, time is of no consequence regarding the life-cycle of the virus, and in some cases a virus's genome may exist for years without a need to activate until the pathogen's genetic programming senses the time is right to initiate the virus's replication process. The only opportunity the immune system may have to combat a latent virus is at the time when copies of the virus leave the host cell and circulate in the blood or tissues, in search of another perspective host cell.

The traditional medical approach to combating infectious agents such as bacteria have limited value in managing or eradicating aggressive viral infections, especially those that are latent viral infections. Synthetic antibiotics, generally used to augment the body's capacity to produce naturally occurring antibodies against bacterial infections, circulate the blood stream for limited periods of time and thus have little success in combating latent viruses that are protected by their host cell. Stimulating the body's immune system recognition by administering a vaccine may have limited value in combating latent viral infections. Vaccines generally are intended to introduce to the body's immune system an attenuated, noninfectious intact bacteria or virus, or pieces of a bacteria or virus so the immune system is able to recognize and process the infectious agent and generate antibodies directed to assist in killing the pathogen. Once the immune system has been primed to recognize an intruder, antibodies are generally produced by B-cells in generous quantities in an effort to repel an invader. Following the initial antibody response, the antibody production diminishes if the body fails to recognize evidence of a substantial ongoing active infection. Latent viruses may lie in wait inside the protective shelter of its host cell and not activate its reproductive cycle until a time where the innate antibody response by the body has declined to the point where it is in fact ineffective in intercepting the viral copies once the viral copies have been released into circulation.

The human immunodeficiency virus demonstrates three factors, which make this pathogen particularly challenging to seek out and eradicate. First: the host for HIV is the T-Helper cell. The T-Helper cell is a key element in the immune system's response since it helps coordinate the body's defensive actions against most pathogens seeking to invade the body's tissues. In cases of a bacterial infection versus a viral infection, T-Helper cells actively direct which immune cells will rev-up and engage the particular infection. Since HIV infects and disrupts T-Helper cells, coordination of the immune response against the virus is disrupted, thus limiting the body's capacity to mount an appropriate and effective response against the presence of HIV and eradicate the virus.

Second: again, latent viruses such as HIV have a strategic advantage. When the immune system first recognizes a pathogen's existence and begins to generate antibodies against a particular pathogen, the response is robust. Once time has passed and the immune system fails to detect an active threat, the production of antibodies against the particular pathogen diminishes. When HIV infects a T-Helper cell, the viral genome lays dormant, sometimes for years before taking command of the T-Helper cell's biologic functions. HIV may therefore generate a very active initial immune response to its presence, but if the virus sits dormant inside T-Helper cells for months or years, the antibody response to the virus will diminish over time. There may not be an adequate quantity of circulating antibodies to actively engage the HIV virions as they migrate from the T-Helper cell that generated the copies to uninfected T-Helper cells that will serve as a new host to support further replication. If the immune system's response is insufficient during the period while the virus is exposed and vulnerable, it becomes extremely difficult for the body to eradicate the virus. In addition, HIV has as its most outer surface an enveloped made from the T-Helper cell's own outer cell membrane; therefore much of the surface of an HIV virion would appear to the immune system to be naturally occurring tissue, not the surface characteristics generally recognizable as a pathogen.

The human immunodeficiency virus posses a third, very elusive mode of action, which the virus actively utilizes to defeat the body's immune system. HIV carries in its genome a segment of genetic material that directs an infected T-Helper cell to create and mount on its surface FasL receptors. T-Helper cells carry, on the surface of their outer cell membrane Fas receptors. A Fas receptor, when triggered, initiates apoptosis in the cell. Apoptosis is a biologic process that causes a cell to terminate itself. A T-Helper cell infected with the HIV virus is therefore capable of killing noninfected T-Helper cells that the infected T-Helper cell encounters as it traverses the body. The occurrence of AIDS is therefore enhanced not only by the number of T-Helper cells that become incapacitated due to direct infection by the HIV virus, but also by the number of noninfected T-Helper cells that are eliminated from circulation by coming in contact with infected T-Helper cells.

Acquired Immune Deficiency Syndrome (AIDS) occurs as a result of the number of circulating T-Helper cells declining to a point where the immune system's capacity to mount a successful response against opportunistic infectious agents is critically compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalovirus or other pathogens, the body becomes overwhelmed with the opportunistic infection and the patient becomes ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The human immunodeficiency virus's outermost shell is referred to as its envelope. HIV locates its host by utilizing probes affixed to the outer surface of the envelope. The HIV virus has at least two types of glycoprotein probes attached to the outer surface of its envelope. HIV utilizes a glycoprotein probe 120 (gp 120) to locate a CD4 cell-surface receptor on a T-Helper cell. Once an HIV gp 120 probe has successfully engaged a CD4 cell surface-receptor on a T-Helper cell a conformational change occurs in the probe and a glycoprotein 41 (gp 41) probe is exposed on HIV's surface. The gp 41 probe's intent is to engage a CXCR4 or CCR5 cell-surface receptor on the same T-Helper cell. Once a gp 41 probe on the HIV virion engages a CXCR4 or CCR5 cell-surface receptor, HIV opens an access port through the T-Helper cell's outer membrane.

Once the virus procures an access port into the T-Helper cell, the HIV virion inserts into the T-Helper cell two positive strand RNA molecules approximately 9500 nucleotides in length. Inserted along with the RNA strands are the enzymes: reverse transcriptase, protease and integrase. Once the virus's genome gains access to the interior of the T-Helper cell, in the cytoplasm, the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA becomes read by a host cell's polymerase molecule and the virus's genetic information commands certain cell functions to carry out the replication process to construct copies of the human deficiency virus.

The outer layer of the HIV virion is comprised of a portion of the T-Helper cell's outer cell membrane. In the final stage of the replication process, as a copy of the HIV internal shell referred to as a capsid, which carries the HIV genome, buds through the host cell's outer membrane, the capsid acquires as its outermost shell, a wrapping of lipid bilayer, which it harvests from the host cell's outer membrane. Vaccines are generally comprised of copies of a particular virus or a bacterium, weakened to the point the pathogen is incapable of causing an infection, or a vaccine is often comprised of pieces of a virus or bacteria. In the case of HIV, since the surface of this pathogen is an envelope comprised of lipid bilayer taken from the host T-Helper cell's outer membrane, a vaccine might not only target HIV virions, but might also have deleterious effects on the T-Helper cell population. Antibodies produced to combat HIV infections may not be able to differentiate between the surface of an infectious HIV virion and a noninfected T-Helper cell, and such antibodies may act to coat and assist in the elimination of both targets. In such a scenario, since the vaccine might cause a decline in the number of available T-Helper cells, it is conceivable that such a vaccine might paradoxically induce clinically apparent AIDS in a non-HIV infected patient whom received such a vaccine.

It is clear that the traditional approach of utilizing antibiotics or providing vaccines to stimulate the immune system to produce endogenous antibodies, by themselves, is an ineffective strategy to manage a virus as elusive and deadly as HIV. Drugs that interfere with the replication process of HIV generally slow progression of the infection by the virus, but do not eliminate the virus from the body nor the threat of the clinical symptoms of AIDS. A new strategy is desperately needed in order to successfully combat HIV and prevent the occurrence of AIDS.

In effect, it has been reported that recent vaccines designed to prevent HIV infections have had limited effect. HIV has been regarded as a pathogen that possess the capacity to create a high rate of genetic mutation and thus copies of the virus can readily adapt features that help it circumvent the effects of the antiviral drugs currently in use to combat the virus. It has been reported that given the length of time HIV infects an individual, new, more resistant strains of the virus will appear in the same patient as a result of the introduction of anti-viral therapies, to the point that single drug therapy intended to slow down the virus's replication process is well recognized ‘not’ to be an effective treatment strategy.

A recent approach to creating a vaccine against HIV has been to take a subset of genetic material from an HIV virion and place this inside an alternative virus, in at least one case the virus ‘adenovirus’ has been used. A weakened form of adenovirus was to be introduced into the body and engaged by the immune system. Once white cells had engulfed the weakened adenovirus, the immune system was to become alerted to the presence of HIV by recognizing the existence of the HIV genetic material carried by the adenovirus. The intent was to stimulate cytotoxic white cells to recognize and kill HIV virions and cell infected by HIV. This innovative approach to managing HIV infections has yet to be proven to be successful. HIV's genetic material is generally never exposed to the surveillance apparatuses of the immune system; though as a result of the function of HIV's genetic material it is conceivable that a cell infected with HIV may demonstrate on its outer surface one or more cell-surface markers that could indicate the cell is infected with the virus. This approach has yet to be found to be practical or effective.

It is understood that the B-cells of the immune system, when activated, are capable of generating antibodies not only directed against viruses, bacteria, cellular parasites, but also ‘foreign proteins’. If an insect or reptile venom, which generally consists of one or more proteins, is injected into a body, and if sufficient time is allowed and the victim does not have a fatal reaction to the venom, the victim's B-cells will generate antibodies against the foreign proteins comprising the venom. Injectable medications that consist of one or more proteins may stimulate the B-cells of the immune system to generate antibodies against the medication. Often protein-based medications, such as insulin, are therefore designed and constructed to appear to the immune system as identical as possible to the naturally occurring human protein it is supplementing. The closer a protein intended to provide a medical treatment, appears to the body like the endogenous protein it is supplementing, in theory, the less likely the medical treatment protein will generate an immune response against the medication.

The unique surface feature of the human immune deficiency virus are, in fact, its surface probes. The remainder of the surface envelope of an HIV virion is comprised of, and appears as, the outer cell membrane of a T-Helper cell. Affixed to the exterior surface of an HIV virion are a quantity of gp 120 probes and gp 41 probes, which represent the distinguishing features of the pathogen.

Since the introduction of a protein alone into the body can create an immune response and cause antibodies to be formed, taking simply the glycoprotein probes, which are unique to HIV might act as the basis of an effective vaccine. The two HIV probes gp 120 and gp 41 are constructed of a combination of protein and lipid structures. Inserting into the body a quantity of intact or partially intact gp 120 and gp 41 probes, like one would insert a traditional vaccine, might produce an immune response by the B-cells directed solely against the presence of these probes. Since the probes only occur on the surface of HIV virions, antibodies created by the B-cells of the immune system specifically against such probes would target HIV virions and not T-Helper cells.

In addition, despite the genetic variation that the HIV virus might undergo, since the T-Helper cell's CD4, CXCR4, and CCR5 cell-surface receptors do not appreciably change their construct over time, it stands to reason the construct of HIV's gp 120 probes and gp 41 probes cannot undergo substantial physical change. HIV's probes need to remain relatively standard in their construct if HIV intends to maintain its capability to successfully access T-Helper cells. Where medications directed at slowing down the virus's replication process may see a decline in their effectiveness due to a defensive genetic variation by the virus, a vaccine made solely of the protein subunit of HIV's gp 120 probes should not diminish in its effectiveness despite HIV's attempt to mutate. The design of HIV's probes is dependent upon the construct of the T-Helper cells' cell-surface receptors. If the T-Helper cells' cell-surface receptors do not change in their physical construct, the construct of the protein subunit of HIV's gp 120 probe cannot be substantially altered otherwise HIV risks becoming incapable of propagating itself.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure receptors that the cell uses to detect properties of the environment surrounding the cell membrane. The cytoplasm acts as a filling medium inside the boundaries of the plasma cell membrane and is comprised mainly of water and nutrients such as amino acids, oxygen, and glucose. The nucleus, organelles, and ribosomes are suspended in the cytoplasm. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information in a mRNA molecule and manufacture proteins to the specifications of the instruction code physically present in the mRNA molecule.

The majority of the deoxyribonucleic acid (DNA) comprises the chromosomes, double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. Some types of RNAs are classified as messenger RNAs (mRNA), transport RNAs (tRNA) and ribosomal RNAs (rRNA).

Proteins are comprised of a series of amino acids bonded together in a linear strand, sometimes referred to as a chain; a protein may be further modified to be a structure comprised of one or more similar or differing strands of amino acids bonded together. Insulin is a protein structure comprised of two strands of amino acids, one strand comprised of 21 amino acids long and the second strand comprised of 30 amino acids, the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Certain forms of glycoproteins are known to be utilized as cell-surface receptors. Messenger RNAs (mRNA) are created by transcription of DNA; they exit the nucleus of the cell, and are utilized as protein manufacturing templates by ribosomes. A ribosome is a protein complex that manufactures proteins by deciphering the instruction code located in a mRNA molecule. When a specific protein is needed, pieces of the ribosome complex bind around the strand of a mRNA that carries the specific instruction code that will generate the required protein. The ribosome traverses the mRNA strand and deciphers the genetic information coded into the sequence of nucleotides that comprise the mRNA molecule.

Transport RNAs (tRNA) are constructed in the nucleus or in the mitochondria, and are coded for one of the 20 amino acids the cells of the human body use to construct proteins. Once a tRNA is created by transcription of the DNA, the tRNA seeks out the type of amino acid it has been coded for and attaches to that specific amino acid. The tRNA then delivers the amino acid it carries to a ribosome that is waiting for that specific amino acid. Proteins are manufactured by the ribosomes binding together sequences of amino acids. The order by which the amino acids are bonded together is dictated by the way the mRNA is constructed and how the ribosome interprets the information encoded in the string of nucleotides present in the mRNA strand.

A sequence of three nucleotides present in a mRNA molecule represents a unit of information referred to as a codon. Codons code for all of the 20 amino acids used to construct protein molecules and also for START and STOP commands. In the process known as translation, the ribosome decodes the codons present in the mRNA, initiating the protein manufacturing process at a START codon, then interfacing with tRNAs carrying the amino acids that match the sequence of codons in the mRNA as the ribosome traverses the length of the mRNA molecule. The ribosome functions as a protein factory by taking amino acids delivered by tRNAs and binding the amino acids together in the order dictated by the sequence of codon instructions coded into the mRNA template as directed by the manner of the nucleic acid arrangement in the mRNA molecule. Protein synthesis ceases when a ribosome encounters a STOP code. The protein molecule is released by the ribosome.

It is well known to the medical scientific community the method of generating proteins for medical treatment purposes. Insulin is a common protein generated for medical treatment purposes. Bacteria, animal cells or hybrid cells comprised of a bacteria-animal cell combination can be utilized as factories to build large quantities of specific proteins. DNA specific for the desired protein can be introduced into the intended factory cell. The factory cell will then decipher the DNA using innate biologic mechanisms and from the DNA, generate mRNA. The mRNA, specific for the desired protein, then acts as a template for the construction of the desired protein. The mRNA is read by ribosomes and the protein molecule is created. The protein subunit of the glycoprotein 120 probe can be generated in large volumes in a similar manner.

HIV's RNA genome consists of two identical positive sense single stranded RNA approximately 9500 nucleotides in length. The core genes comprising the RNA genome consist of the genes known as Gag, Pro, Pol and Env. The Gag gene translates into molecules of capsid proteins. The Pro gene translates into protease molecules. The Pol gene translates into molecules of reverse transcriptase. The Env gene translates into the envelope proteins. The Env gene carries the instruction coding for the amino acid sequence and physical construct characteristics, in RNA format, for the molecular structure of the protein subunit of the glycoprotein 120 probe.

### BRIEF SUMMARY OF THE INVENTION

A medical treatment device comprised of a quantity of protein subunits of the glycoprotein 120 probe, these protein subunits of the glycoprotein 120 probes similar in construct to the protein subunit of the glycoprotein 120 probes found on the surface of a naturally occurring human immunodeficiency virus virion generally known to the medical scientific community as the glycoprotein 120 probe. These protein subunits of the glycoprotein 120 probe are to be held in suspension in a hypoallergenic medium formulated to keep intact the protein subunits of the glycoprotein 120 probe, intended to be introduced into the body, whereby the introduction of the protein subunits of the glycoprotein 120 probe is intended to stimulate the production of antibodies against the protein subunit of glycoprotein 120 probes with the intention to generate a defensive immune response by the body in the form of antibodies directed against the presence of the human immunodeficiency virus. The objective of the vaccine to cause the body to generate antibodies intended to engage the protein subunit of the glycoprotein 120 probe and only the protein subunit of the glycoprotein 120 probe, as it is affixed to the surface of a HIV virion, rendering the HIV virion unable to attach to a host cell by blocking the protein subunit of the glycoprotein 120 probes. By generating an antibody to engage and block the protein subunit of the glycoprotein 120 probes as they exist on the surface of a HIV virion, this would cause the HIV virions to be unable to engage CD4 T-Helper cells, thus incapable of further infecting cells in the body. By generating an antibody to engage and only engage the protein portion of the glycoprotein 120 probe will produce a safe, practical, and effective vaccine to prevent and treat those at risk for developing AIDS.

### DETAILED DESCRIPTION OF THE INVENTION

Vaccines created to combat infectious medical diseases are generally comprised of a liquid medium that carries in suspension intact virus or bacteria of a particular strain that has been weakened to the point it is unable to generate an infection, or a vaccine is comprised of pieces of a virus or bacteria of a particular strain. The weakened, but intact virus or bacteria or the pieces of a virus or bacteria are meant to activate the immune system of the individual receiving the vaccine, such that the individual's immune system will generate antibodies against the intact virus or bacteria or the portions of the virus or bacteria the immune system is exposed to by way of the vaccine.

Due to the fact that the exterior surface of a human immunodeficiency virus is comprised of essentially the same material as the outer membrane of a T-Helper cell, introducing to the immune system weakened HIV virions or portions of HIV's exterior envelope may lead to a vaccine that has deleterious effects on the individual's T-Helper cell population. Antibodies generated by the immune system directed against the surface characteristics of a T-Helper cell may lead to antibodies seeking out and engaging endogenous T-Helper cells, rendering these endogenous T-Helper cells ineffective. If the antibodies generated by such a vaccine were robust enough in their action, the vaccine could lead to generating an AIDS-like clinical picture due to a reduction in the number of effective circulating T-Helper cells.

A vaccine that introduced portions of the internal structures of the HIV virion would generally be ineffective since the internal structures of HIV are never exposed to the immune system as HIV virions traverse the body in search of a host cell.

Vaccines are generally only effective if they are targeted against a structure that is specific to the pathogen and a structure that is readily exposed to the immune system and vulnerable to the immune system's defense mechanisms. The unique identifiers of the human immunodeficiency virus are the two known glycoprotein probes that exist on the surface of HIV's virion. The glycoprotein 120 probe and the glycoprotein 41 probe are unique to HIV, and are the structures on the surface of HIV's virion that the virus utilizes to seek out and engage a perspective T-Helper cell host. The glycoprotein 41 probe is usually hidden by the glycoprotein 120 probe until the glycoprotein 120 probe has engaged a CD4 cell-surface receptor. Once the glycoprotein 120 probe engages a CD4 cell-surface receptor on a T-Helper cell, a conformational change occurs in the 120 probe and the underlying glycoprotein 41 probe is exposed. The exposed glycoprotein 41 probe is then capable of engaging a CXCR4 or CCR5 cell-surface receptor present on the T-Helper cell.

Glycoproteins are generally comprised of two subunits, a lipid molecular subunit and a protein molecular subunit. The lipid portion of the glycoprotein acts as an anchor to hold the glycoprotein structure fixed into the bilayer lipid envelope that comprises the surface the of the HIV virion. The protein subunit of the glycoprotein probe extends out from the bilayer lipid envelope and acts to engage a cell-surface receptor on the surface of a prospective host cell. The protein portion of the glycoprotein is generally what would be exposed on the surface of an HIV virion and would be the unique identifier of an HIV virion.

Antibodies are generated by the immune system in response to the presence of a virus, bacteria, or foreign substance identified in the fluids or tissues within the outer boundaries of the body. Antibodies are intended to act to compromise the outer surface of a pathogen, or antibodies are to coat the exterior of a virus, bacteria or foreign substance to make the pathogen or foreign substance easier for the immune system to identify and clear from the body. Antibodies may differ in construction and function depending upon the virus, bacteria or foreign substance identified by the immune system. Further, antibodies may differ in construction and function depending upon the portion of the virus, portion of the bacteria or portion of the foreign substance presented to and identified by the immune system.

A vaccine comprised of intact glycoprotein 120 probes or intact glycoprotein 41 probes may be ineffective in stimulating the necessary immune response due the antibodies may not be constructed properly to engage the working portion of the glycoprotein probes, specifically the protein subunit of the glycoprotein 120 probe due to the fact this is the unique identifier of HIV and the protein portion of the glycoprotein 120 probe is the only portion of the HIV probes that is generally exposed. A vaccine comprised of only the protein subunit of the glycoprotein 120 probe is necessary to act as an effective stimulant of the immune system. Such a vaccine can be effective only if not accompanied by any other portion of the HIV virion. It is critical to the effectiveness and success of the described vaccine that no portion of the outer envelope of the HIV virion other than the protein portion of the glycoprotein 120 probe accompany the protein subunits of the glycoprotein 120 probe when introduced into the body as a vaccine. In addition, no portion of the glycoprotein 120 probe other than the protein subunit accompany the protein subunits of the glycoprotein 120 probe when introduced into the body as a vaccine.

A vaccine to prevent and treat HIV infections would be comprised of a quantity of protein subunits of the glycoprotein 120 probe, these protein subunits of the glycoprotein 120 probe being similar in construct to the protein subunit of the glycoprotein 120 probes found on the surface of a naturally occurring human immunodeficiency virus virion. These protein subunits of the glycoprotein 120 probe being utilized as a vaccine, would be held in suspension in a hypoallergenic medium intended to be introduced into the body, whereby the introduction of the protein subunits of the glycoprotein 120 probe is intended stimulate the production of antibodies against the protein subunit of the glycoprotein 120 probes to generate a defensive immune response by a body against the presence of the human immunodeficiency virus while not generating an immune response to the hypoallergenic medium.

The mechanism to produce protein molecules of a particular design is well known to the medical community. Bacteria or animal cells or hybrid cells can be readily fashioned to act as biologic factories to produce large quantities of proteins of a particular construct. Inside a cell utilized to act as a factory, messenger RNA designed to construct the protein subunit of the glycoprotein 120 probe can interact with ribosomes to produce quantities of the protein. The instruction coding for the messenger RNAs to be used is readily available in the HIV RNA genome in the location of the Env gene. HIV's Env gene carries the instruction coding for the amino acid sequence and physical construct characteristics of the molecular structure of the protein subunit of the glycoprotein 120 probe. Once such protein subunits of the glycoprotein 120 probe is produced in quantity by bacteria, animal cells, or hybrid cells, an appropriate quantity would be placed in a hypoallergenic suspension medium to be used as a vehicle to deliver the quantity of these protein subunits of the glycoprotein 120 probe into the body.

The hypoallergenic suspension is meant to act as a medium to facilitate delivery of the protein subunits of the glycoprotein 120 probe into the body. The hypoallergenic suspension is meant specifically not to generate an immune response so that the immune system will direct its sole response against the protein subunits of the glycoprotein 120 probe suspended in the medium. The hypoallergenic suspension medium is meant to act to preserve the structure of the protein subunits of the glycoprotein 120 probe prior to and during delivery of the hypoallergenic suspension into the body. The hypoallergenic suspension medium containing the quantity of protein subunits of the glycoprotein 120 probe may be injected into the body, or may be administered by an oral route or administered by a rectal route or administered by a vaginal route in women or aerosolized and inhaled.

What is claimed:

1. A medical treatment device comprised of a quantity of protein subunits of the glycoprotein 120 probe molecular structure, said protein subunits similar in construct to the intact protein portion of the glycoprotein 120 probes found on the surface of a naturally occurring human immunodeficiency virus virion, wherein said protein subunits are held in suspension in a hypoallergenic medium intended to be introduced into a living body, whereby the introduction of said protein subunits suspended in the hypoallergenic medium is intended to stimulate solely the production of antibodies against said protein subunits with the intention to generate an immune response by said living body against the human immunodeficiency virus, the said antibodies solely to engage the protein subunits as they exist on the surface of human immunodeficiency virus virion as to render the virion's protein subunit of the glycoprotein 120 probes ineffective in their capacity to properly engage a CD4 cell-surface receptor located on a T-Helper cell.

2. The medical treatment device in claim 1 whereby the quantity of protein subunits of the glycoprotein 120 probe suspended in a hypoallergenic medium is introduced into the body by transmission means whereby an oral form is delivered by an oral route.

3. The medical treatment device in claim 1 whereby the quantity of protein subunits of the glycoprotein 120 probe suspended in a hypoallergenic medium is introduced into the body by a transmission means whereby an aerosolized form is delivered by an inhaled route.

4. The medical treatment device in claim 1 whereby the quantity of protein subunits of the glycoprotein 120 probe suspended in a hypoallergenic medium is introduced into said body by transmission means of an injectable method whereby the dermis of said body is penetrated by way of a sharp hollow instrument such a needle and the quantity of protein subunits of the glycoprotein 120 probes suspended in a hypoallergenic medium is deliver into the body by way of passing through said sharp hollow instrument.

5. The medical treatment device in claim 1 whereby the quantity of protein subunits of the glycoprotein 120 probe suspended in a hypoallergenic medium is introduced into the body by transmission means of a rectal suppository.

6. The medical treatment device in claim 1 whereby the quantity of protein subunits of the glycoprotein 120 probe suspended in a hypoallergenic medium is introduced into the body by transmission means of a vagina suppository.

7. The medical treatment device in claim 1 wherein the body is comprised of the physical structures of a human being.
